(19) United States
(12) Patent Application Publication
(10) Pub. No.: US 2009/0150088 A1
(43) Pub. Date: Jun. 11, 2009

(54) METHOD OF ANALYZING BEHAVIOR OF POLLUTANTS THROUGH PREDICTION OF TRANSVERSE DISPERSION COEFFICIENT USING BASIC HYDRAULIC DATA IN STREAM

(76) Inventors: Il-Won SEO, Seoul (KR); Tae-Myoung JEON, Seoul (KR); Kyung-Oh BAEK, Anyang-si (KR)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/023,666

(22) Filed: Jan. 31, 2008

(30) Foreign Application Priority Data
Dec. 6, 2007 (KR) 10-2007-0126317

(51) Int. Cl. G01N 33/00; G06F 19/00

(52) U.S. Cl. 702/25

(57) ABSTRACT

Disclosed herein is a method of analyzing the behavior of pollutants in a stream through the prediction of a transverse dispersion coefficient. The method includes the steps of (a) surveying and storing stream data, including the flow velocity, depth, sinuosity, width and longitudinal dispersion coefficient of a target stream; (b) deriving a transverse dispersion coefficient by arranging only dimensionless factors that influence transverse mixing through dimensional analysis, and assuming that the transverse dispersion coefficient is a product of power functions; (c) collecting hydraulic data and transverse dispersion coefficient data of domestic and foreign streams; (d) deriving the predicted value of the transverse dispersion coefficient from the transverse dispersion coefficient through regression analysis; (e) obtaining a numerical solution by constructing a numerical model using the flow velocity, depth, sinuosity, width and longitudinal dispersion coefficient of the stream and the transverse dispersion coefficient.

### BACKGROUND OF THE INVENTION

1. Field of the Invention

The present invention relates generally to a method of analyzing the behavior of pollutants through the prediction of a transverse dispersion coefficient using basic hydraulic data in a stream, and, more particularly, to a method of analyzing the behavior of pollutants through the prediction of a transverse dispersion coefficient, which enables a user having no observed transverse dispersion coefficient data to conveniently predict a transverse dispersion coefficient using only basic hydraulic data and to effectively use the predicted transverse dispersion coefficient so as to analyze the behavior of pollutants, thus providing basic data for the operation of a water intake facility and the development of a water quality prediction and warning system.

2. Description of the Related Art

In the case of natural streams, the process of the transport and dispersion of pollutants is complex due to the non-uniformity of a flow velocity structure and the development of secondary flow, attributable to meandering, the development of a dead-zone, the irregularity of a riverbed, and a structure including pools and riffles.

When the mixing of pollutants in such a natural stream is analyzed using a two-dimensional (2D) model, a longitudinal dispersion coefficient and a transverse dispersion coefficient are used as parameters. Since such dispersion coefficients are representative factors that are used to determine the extent of mixing of pollutants in a stream, special attention must be paid to the process of the determination of the dispersion coefficients.

In general, methods of determining dispersion coefficients in the analysis of the spread of pollution in a stream may be classified into observation methods that use concentration data that are acquired through tracer experiments, and prediction methods that predict dispersion coefficients based on basic hydraulic data. The prediction methods, in turn, may be classified into theoretical equation methods, in which dispersion coefficients are theoretically derived in consideration of the physical mechanism of a shear flow, which causes dispersion, and empirical equation methods, in which dispersion coefficients are acquired through regression analysis based on a plurality of pieces of experimental data. However, since the complexity of a theoretical equation is simplified using empirical methodology, or an empirical equation may be developed based on a theoretical background, the two methodologies are not contradictory to each other, but are complementary to each other.

Accordingly, when a transverse dispersion coefficient, determined based on a concentration distribution curve observed by carrying out tracer experiments in a stream, exists, it can be input into the numerical model. However, since transverse dispersion coefficients have not been observed for most Korean streams, it is common to input dispersion coefficients, predicted using theoretical or empirical equations, into numerical models.

Meanwhile, in 1959, in order to perform 2D pollution dispersion analysis, Elder theoretically derived a longitudinal dispersion coefficient on the assumption that the vertical distribution of a main flow was a logarithmic distribution. Since the equation developed by him has a theoretical background and is expressed using simple constants, it has been universally adopted for the determination of the longitudinal dispersion coefficient.

However, the universal use of a plurality of existing empirical equations for predicting transverse dispersion coefficients, which have been proposed based on the geographical and hydraulic factors of streams so as to predict the transverse dispersion coefficients in the case where no concentration dispersion data has actually been measured for the streams, entails many errors because the empirical equations cannot appropriately represent the meandering characteristics of streams, and were derived based on data for specific streams. As a result, the provision of a universal transverse dispersion coefficient empirical equation, which can be applied to the various geographical and hydraulic conditions of streams, is required.

### SUMMARY OF THE INVENTION

Accordingly, the present invention has been made keeping in mind the above problems occurring in the prior art, and an object of the present invention is to provide a method that can be effectively used for the analysis of the behavior of pollutants using a universal transverse dispersion coefficient equation that can be applied to streams having various geographical and hydraulic conditions using only basic hydraulic data in a statistical manner in the case where there is no observed transverse dispersion coefficient at the time of predicting a transverse dispersion coefficient, which is a required parameter for the 2D analysis of the mixing of pollutants when the pollutants flow into a stream.

In order to accomplish the above object, the present invention provides a method of analyzing the behavior of pollutants in a stream through the prediction of a transverse dispersion coefficient, the method including the steps of (a) surveying and storing stream data, including the flow velocity, depth, sinuosity, width and longitudinal dispersion coefficient of a target stream; (b) deriving a transverse dispersion coefficient by arranging only dimensionless factors that influence transverse mixing in a natural stream through dimensional analysis, and assuming that the transverse dispersion coefficient is a product of power functions; (c) collecting hydraulic data and transverse dispersion coefficient data of domestic and foreign streams, which are required for development of an empirical equation for predicting the transverse dispersion coefficient; (d) deriving the predicted value of the transverse dispersion coefficient from the transverse dispersion coefficient of step (b) through regression analysis based on the data collected at step (c); and (e) obtaining a numerical solution, that is, a concentration of the pollutants in the stream, by constructing a numerical model using the flow velocity, depth, sinuosity, width and longitudinal dispersion coefficient of the stream, stored at step (a), and the transverse dispersion coefficient, obtained at step (d), as input data.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a diagram showing the results of comparison between the predicted and observed values of the transverse dispersion coefficient according to the present invention; and

FIG. 2 is a flowchart showing an embodiment for analyzing the behavior of pollutants through the prediction of the transverse dispersion coefficient according to the present invention.

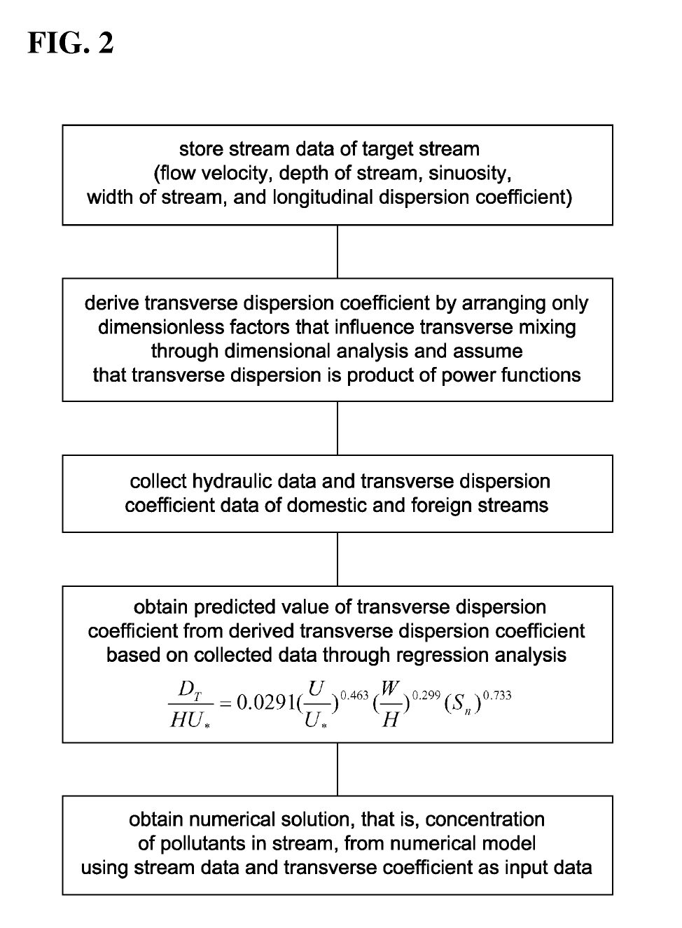

### DESCRIPTION OF THE PREFERRED EMBODIMENTS

Reference now should be made to the drawings, in which the same reference numerals are used throughout the different drawings to designate the same or similar components.

In the present invention, when, in order to develop a new empirical equation for predicting a transverse dispersion coefficient, only dimensionless factors, which considerably influence transverse mixing in natural streams in dimensional analysis, are arranged and the transverse dispersion coefficient is then derived therefrom, the following Equation 1 is obtained:

$$\frac{{D}_{T}}{{HU}_{*}} = f ( {S}_{n} , \frac{U}{{U}_{*}} , \frac{W}{H} ) \qquad ( 1 )$$

where D_{T} is the transverse dispersion coefficient, H is the average depth of water, U_{*} is the shear flow velocity, f is an arbitrary function, Sₙ is the sinuosity, U is the average flow velocity in a flow direction, and W is the width of a stream.

Thereafter, in order to develop the empirical equation through regression analysis, it is assumed that the Equation 1 is a product of power functions, as shown in the following Equation 2:

$$\frac{{D}_{T}}{{HU}_{*}} = {{a}_{0} ( {S}_{n} )}^{{a}_{1}} {( \frac{U}{{U}_{*}} )}^{{a}_{2}} {( \frac{W}{H} )}^{{a}_{3}} \qquad ( 2 )$$

where a₀, a₁, a₂, and a₃ are regression constants.

Thereafter, in the present invention, in order to develop an empirical equation for predicting a transverse dispersion coefficient, hydraulic data and transverse dispersion coefficient data (6 pieces of domestic data and 26 pieces of foreign data) were collected at 32 points in domestic and foreign streams. Since it is unsuitable for the realization of universality to develop a transverse dispersion coefficient empirical equation using only domestic stream data, foreign data was also collected. Cases where the sinuosity could be accurately determined were selected as the foreign data from among collected foreign tracer experiment cases.

Meanwhile, the domestic data and foreign data used in the present invention are listed in the following Table 1. In the following table, containing the collected domestic and foreign data, the shaded portions are 16 pieces of data used for the verification of the developed empirical equation, while the remaining portions are 16 pieces of data that were used for the development of the empirical equation.

TABLE 1

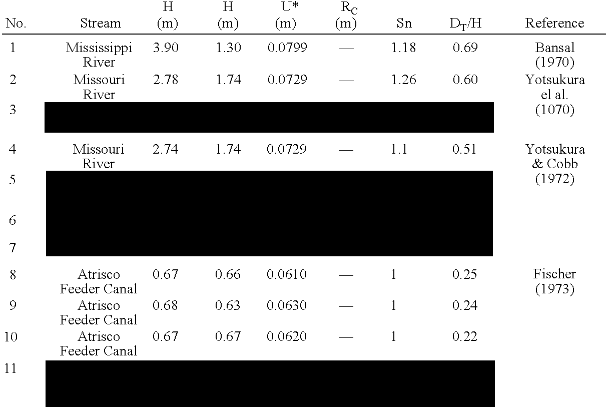

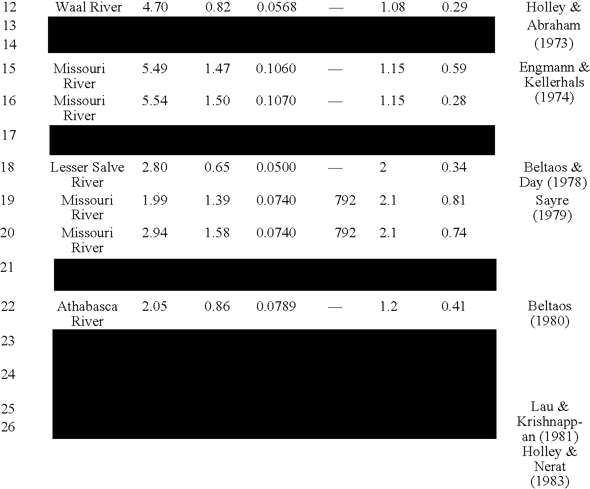

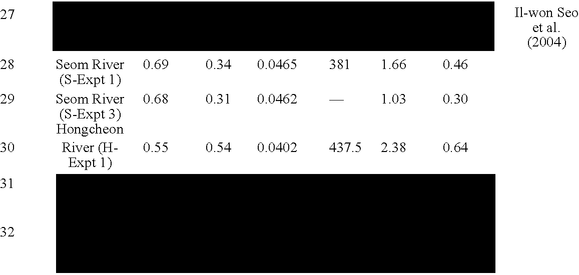

In Table 1, the classification of data used for the development and verification of the empirical equation was randomly performed, but was performed such that the distributions of discrepancy rates of two data groups, which were used for the development and verification of the empirical equation, are similar to each other.

When the regression constants of Equation 2 are determined based on 16 pieces of collected data, used for the development of the empirical equation, using a Robust regressing method, a final empirical equation is derived, as shown in the following Equation 3:

$$\frac{{D}_{T}}{{HU}_{*}} = 0.0291 {( \frac{U}{{U}_{*}} )}^{0.463} {( \frac{W}{H} )}^{0.299} {( {S}_{n} )}^{0.733} \qquad ( 3 )$$

Meanwhile, a typical regression equation uses a method of minimizing the residual sum of squares using a least squares method. However, this least squares method has a disadvantage in that the influence of outliers is very high because a residual thereof is squared. Accordingly, the regression analysis of the present invention uses the Robust regression method, as described above. This Robust regression method uses a double square weight, and is a method capable of minimizing the influence of outliers in such a way as to first perform regression analysis using a weighted least squares method and then calculate a corrected residual.

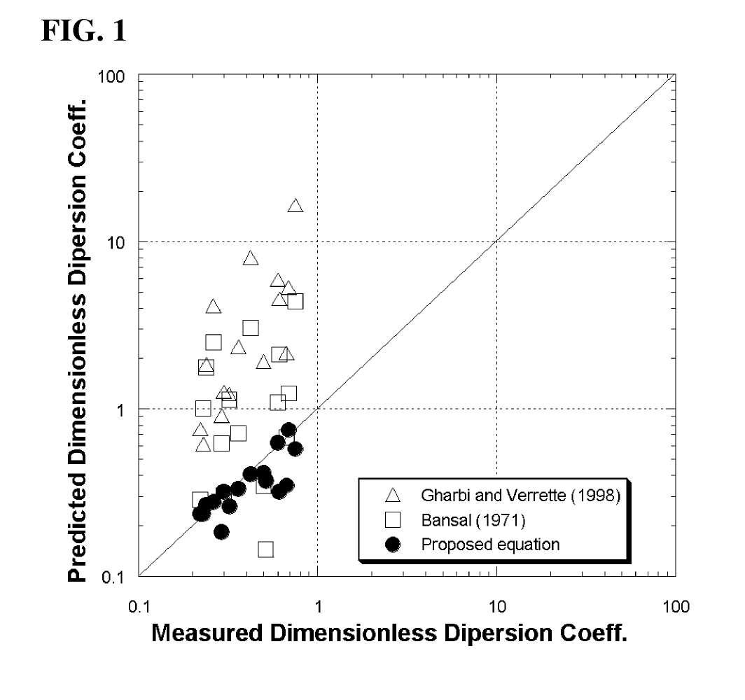

Additionally, in order to verify the developed empirical equation, the results of comparison between Equation 3 and existing studies (Bansal (1971), and Gharbi and Verrette (1998)) using the 16 pieces of verification data of Table 1 are shown in FIG. 1.

From FIG. 1, it can be seen that, when the empirical equation proposed in the present invention is used, the predicted value of the dimensionless transverse dispersion coefficient coincides well with the observed value thereof. In contrast, the empirical equations of the existing studies generally tend to overestimate transverse dispersion coefficients. Therefore, it can be seen that the empirical equation developed in the present invention is effective in predicting an accurate transverse dispersion coefficient.

Meanwhile, in order to analyze the mixing of pollutants in a natural stream, it is preferable to use a precise three-dimensional (3D) analysis model. However, the use of the precise 3D analysis model requires excessive effort and time, and mixing in the direction of the depth of water in most streams occurs rapidly compared to mixing in the transverse and longitudinal directions. Accordingly, when a 2D advection-dispersion model, obtained by integrating a 3D advection-diffusion model over the depth of water, is used, the phenomenon of the dispersion of pollutants can be effectively analyzed.

Meanwhile, so far, in Korea, a 1D longitudinal dispersion model has been used in the practice of stream analysis on the assumption that mixing in the width direction of a stream has been completed. However, in the present invention, in view of the characteristics of Korean streams, in which a pollution source and a water intake facility coexist in the same region, a 2D advection-dispersion equation, such as Equation 4, is used as a governing equation in order to more accurately analyze the behavior of pollutants in the stream plane:

$$\frac{\partial C}{\partial t} + u \frac{\partial C}{\partial x} + v \frac{\partial C}{\partial y} = \frac{1}{h} \frac{\partial }{\partial x} ( h {D}_{L} \frac{\partial C}{\partial x} ) + \frac{1}{h} \frac{\partial }{\partial y} ( h {D}_{T} \frac{\partial C}{\partial y} ) \qquad ( 4 )$$

where C is the concentration of pollutants at an arbitrary time and an arbitrary location, u is the longitudinal flow velocity, v is the transverse flow velocity, h is the depth of water, D_{L} is the longitudinal dispersion coefficient, and D_{T} is the transverse dispersion coefficient.

In this case, flow velocities u and v, the depth of water h, and the longitudinal dispersion coefficient D_{L} and the transverse dispersion coefficient D_{T}, which are the parameters of the model that is determined using the above-described method, are input as input data, with the result that Equation 4 has a single unknown quantity, so that the partial differential equation can be solved.

However, since it is impossible to analytically obtain the concentration C by applying Equation 4 to a domain having complex boundary conditions (a typical natural stream), a numerical model must be constructed, and an approximate solution (a numerical solution) must be generally obtained.

As a result, in the present invention, a typical Finite Difference Method (FDM) and a typical Finite Element Method (FEM) may be used as methods for obtaining numerical solutions.

For reference, the FDM is a method of obtaining a numerical solution by approximating the partial differential equation, which is a governing equation, as a difference equation using a Taylor series. The FDM can more directly obtain solutions, but is difficult to apply to complex domains. The FEM is a method of obtaining a solution by dividing a target domain into a finite number of regions (elements), determining a node representative of each region and approximating the governing equation of the node as simultaneous linear equations. The FDM has a disadvantage in that the computational load increases as the number of simultaneous equations increases in inverse proportion to the size of the elements, but has an advantage in that it can flexibly deal with complex geographies.

The above-described method of analyzing the behavior of pollutants through the prediction of a transverse dispersion coefficient using basic hydraulic data in a stream provides an effect of enabling a user having no observed transverse dispersion coefficient data to conveniently predict a transverse dispersion coefficient using only basic hydraulic data and to effectively use the predicted transverse dispersion coefficient so as to analyze the behavior of pollutants, thus providing basic data for the operation of a water intake facility and the development of a water quality prediction and warning system.

Although the preferred embodiments of the present invention have been disclosed for illustrative purposes, those skilled in the art will appreciate that various modifications, additions and substitutions are possible, without departing from the scope and spirit of the invention as disclosed in the accompanying claims.

What is claimed is:

1. A method of analyzing behavior of pollutants in a stream through prediction of a transverse dispersion coefficient, the method comprising the steps of:
(a) surveying and storing stream data, including a flow velocity, depth, sinuosity, width and longitudinal dispersion coefficient of a target stream;
(b) deriving a transverse dispersion coefficient by arranging only dimensionless factors that influence transverse mixing in a natural stream through dimensional analysis, and assuming that the transverse dispersion coefficient is a product of power functions;
(c) collecting hydraulic data and transverse dispersion coefficient data of domestic and foreign streams, which are required for development of an empirical equation for predicting the transverse dispersion coefficient;
(d) deriving the following equation from the transverse dispersion coefficient of step (b) through regression analysis based on the data collected at step (c), and obtaining a predicted value of the transverse dispersion coefficient:
$$\frac{{D}_{T}}{{HU}_{*}} = 0.0291 {( \frac{U}{{U}_{*}} )}^{0.463} {( \frac{W}{H} )}^{0.299} {( {S}_{n} )}^{0.733}$$
where D_{T} is the transverse dispersion coefficient, H is an average depth of water, U_{*} is a shear flow velocity, Sₙ is a sinuosity, U is an average flow velocity in a flow direction, and W is a width of the stream; and
(e) obtaining a numerical solution, that is, a concentration of the pollutants in the stream, by constructing a numerical model using the flow velocity, depth, sinuosity, width and longitudinal dispersion coefficient of the stream, stored at step (a), and the transverse dispersion coefficient, obtained at step (d), as input data.

2. The method as set forth in claim 1, wherein:
the transverse dispersion coefficient, derived by arranging only the dimensionless factors at step (b), is
$$\frac{{D}_{T}}{{HU}_{*}} = f ( {S}_{n} , \frac{U}{{U}_{*}} , \frac{W}{H} ) ;$$
the assumed product of power functions is
$$\frac{{D}_{T}}{{HU}_{*}} = {{a}_{0} ( {S}_{n} )}^{{a}_{1}} {( \frac{U}{{U}_{*}} )}^{{a}_{2}} {( \frac{W}{H} )}^{{a}_{3}} ,$$
where a₀, a₁, a₂, and a₃ are regression constants; and
a governing equation for obtaining the concentration of the pollutants at step (e) is
$$\frac{\partial C}{\partial t} + u \frac{\partial C}{\partial x} + v \frac{\partial C}{\partial y} = \frac{1}{h} \frac{\partial }{\partial x} ( h {D}_{L} \frac{\partial C}{\partial x} ) + \frac{1}{h} \frac{\partial }{\partial y} ( h {D}_{T} \frac{\partial C}{\partial y} ) ,$$
where C is the concentration of the pollutants at an arbitrary time and an arbitrary location, u is a longitudinal flow velocity, v is a transverse flow velocity, h is the depth of water, D_{L} is a longitudinal dispersion coefficient, and D_{T} is a transverse dispersion coefficient.

3. The method as set forth in claim 1, further comprising the step of verifying the transverse dispersion coefficient obtained at step (d) based on the collected domestic and foreign stream data.
